(19) United States
(12) Patent Application Publication
(10) Pub. No.: US 2020/0029859 A1
(43) Pub. Date: Jan. 30, 2020

(54) BREATH MEASUREMENT DEVICE

(71) Applicant: PHC HOLDINGS CORPORATION, Tokyo (JP)

(72) Inventors: Toru KAWAMOTO, Ehime (JP); Tsuyoshi SAWADA, Ehime (JP); Norihiko TAMAI, Ehime (JP)

(21) Appl. No.: 16/486,470

(22) PCT Filed: Dec. 25, 2017
(86) PCT No.: PCT/JP2017/046372

§ 371 Date: Aug. 15, 2019

(30) Foreign Application Priority Data
Feb. 15, 2017 (JP) 2017-025845

(51) Int. Cl. A61B 5/087; G01N 11/08; G01N 33/497

(52) U.S. Cl. CPC A61B 5/087; G01N 33/497; G01N 11/08

(57) ABSTRACT

A flow rate detector of an exhalation measurement device includes: a pipe body having a flow inlet at a first end into which exhalation flows, and a flow outlet at a second end out of which exhalation flows; and a differential pressure sensor connected to the pipe body. The pipe body includes: a partition gasket which partitions the inside of the pipe body into a flow-in space of exhalation and a flow-out space of exhalation; a connection aperture being provided at the flow-in space and connecting the differential pressure sensor to the flow-in space; a connection aperture being provided at the flow-out space and connecting the differential pressure sensor to the flow-out space; and an air duct of an elongated shape, extending through the partition gasket and allowing the flow-in space and the flow-out space to communicate with each other.

### TECHNICAL FIELD

The present invention relates to, for example, an exhalation measurement device to be used when e.g. detecting asthma, or checking pulmonary function.

### BACKGROUND ART

A conventional exhalation measurement device would include: a measurement apparatus main body into which exhalation is blown; a chamber to temporarily retain the exhalation that has been blown into the measurement apparatus main body; a pump to allow the exhalation within the chamber to be supplied to a measurement section; a control section to control operation of the pump; and a flow rate detector to detect a flow rate of the exhalation that is supplied by the pump.

In other words, when measuring the ammonia or the like that is contained within exhalation, after the exhalation is once retained in the chamber, this exhalation in the chamber is sucked out by the pump, so as to be supplied to the measurement section.

In order to take a measurement of this exhalation, the exhalation needs to be supplied to the measurement section with a predetermined flow rate (i.e., an amount of exhalation to flow per unit time). Thus, as the flow rate detector detects the flow rate of the exhalation that is supplied by the pump, the pump may be controlled on the basis of this detected value, whereby the exhalation is supplied to the measurement section with a predetermined flow rate (see, for example, Patent Document 1).

### SUMMARY OF INVENTION

### Technical Problem

[Patent Document 1] Japanese National Phase PCT Laid-Open Publication No. 2010-509586

### Solution to Problem

A problem in the above conventional example is that the measurement accuracy for exhalation may become lower.

Specifically, the flow rate detector to detect a flow rate of the exhalation that is supplied by the pump measures a pressure of the exhalation at the upstream side and at the downstream side of the exhalation, and detects a flow rate of the exhalation by using a pressure difference therebetween.

However, usually, such a pressure difference in the exhalation is so small that it is difficult to measure the pressure difference in the exhalation with a sufficient accuracy, and this has led to a low detection accuracy of the flow rate of the exhalation that is detected by using the pressure difference.

Thus, when the detection accuracy for the flow rate of the exhalation is low, the pump cannot be appropriately controlled, so that exhalation cannot be supplied to the measurement section with a predetermined flow rate, thereby resulting in a lower measurement accuracy for exhalation.

Accordingly, an objective of the present invention is to enhance the measurement accuracy for exhalation.

In order to attain this objective, an exhalation measurement device according to the present invention includes: a measurement apparatus main body into which exhalation is to be blown; a measurement section to take a measurement of the exhalation; a chamber to temporarily retain the exhalation that has been blown into the measurement apparatus main body; a pump to allow the exhalation in the chamber to be supplied to the measurement section; a control section to control operation of the pump; and a flow rate detector to detect a flow rate of the exhalation that is supplied by the pump to the measurement section.

The flow rate detector includes: a pipe body having a flow inlet at a first end, into which the exhalation flows, and a flow outlet at a second end opposite to the first end, out of which the exhalation flows; and a differential pressure sensor connected to the pipe body.

The pipe body includes: a partitioning member which partitions an inside of the pipe body into a first space at a flow-in side of the exhalation and a second space at a flow-out side of the exhalation; a first connection aperture being provided at the first space side and connecting the differential pressure sensor to the first space; and a second connection aperture being provided at the second space side and connecting the differential pressure sensor to the second space; and an air duct of an elongated shape extending through the partitioning member and allowing the first space and the second space to communicate with each other.

### Effects of Invention

With an exhalation measurement device according to the present invention, a pipe body of a flow rate detector includes a partitioning member which partitions the inside into a first space at a flow-in side of the exhalation and a second space at a flow-out side of the exhalation, and an air duct of an elongated shape extending through the partitioning member and allowing the first space and the second space to communicate with each other. Therefore, in the inside of the pipe body, the exhalation passes through the thin and long air duct.

This induces an increased pressure difference in the exhalation between the first space and the second space, which will be measured with a sufficient accuracy. Therefore, the detection accuracy for the flow rate of the exhalation as detected by using the pressure difference can be enhanced.

As a result of this, the pump to adjust the flow rate of the exhalation is appropriately controlled, and the exhalation is supplied to the measurement section with a predetermined flow rate, whereby the measurement accuracy for exhalation can be enhanced.

### BRIEF DESCRIPTION OF DRAWINGS

FIG. 1 A perspective view showing the construction of an exhalation measurement device according to Embodiment 1 of the present invention.

FIG. 2 FIG. 1 is a control block diagram of the exhalation measurement device.

FIG. 3 FIG. 1 is an exploded perspective view of a flow rate detector of the exhalation measurement device.

FIG. 4 An upper plan view of the flow rate detector of the exhalation measurement device in FIG. 1.

FIG. 5 A side view of the flow rate detector of the exhalation measurement device in FIG. 1.

FIG. 6 A cross-sectional view, taken at line A-A and viewed in the direction of arrows in FIG. 4.

FIG. 7 A cross-sectional view, taken at line B-B and viewed in the direction of arrows in FIG. 5.

FIG. 8 A cross-sectional view showing the construction of a main portion of the exhalation measurement device in FIG. 1.

### DESCRIPTION OF EMBODIMENTS

Hereinafter, with reference to the drawings as necessary, embodiments will be described in detail. Note however that unnecessarily detailed descriptions may be omitted. For example, detailed descriptions of what is well known in the art or redundant descriptions of what is substantially the same constitution may be omitted.

This is to avoid lengthy description, and facilitate the understanding of those skilled in the art.

The accompanying drawings and the following description, which are provided by the present inventors so that those skilled in the art can sufficiently understand the present disclosure, are not intended to limit the scope of claims.

Hereinafter, an embodiment of the present invention will be described with reference to the drawings.

### (Embodiment 1)

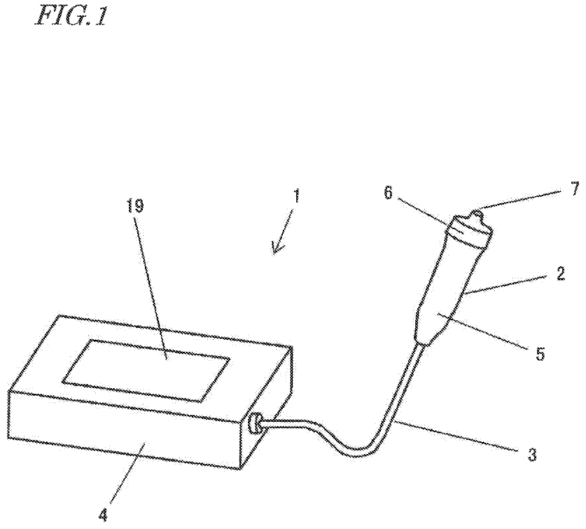

FIG. 1 shows an exhalation measurement device 1 to be used when e.g. detecting asthma, or checking pulmonary function.

The exhalation measurement device 1 according to the present embodiment measures an amount of nitrogen monoxide that is contained in exhalation (i.e., a nitrogen monoxide concentration within exhalation), for example. In the exhalation measurement device 1, a handle section 2 with which to perform sucking and blowing of exhalation is connected to the measurement apparatus main body 4 via a tube 3.

The handle section 2 includes a handle section main body 5 and a mouthpiece 6 that is mounted above the handle section main body 5.

As a user sucks in for exhalation, with his or her mouth being placed on an exhalation opening 7 of the mouthpiece 6, atmospheric air is taken into the handle section main body 5 through an intake port (not shown). From the atmospheric air that has been taken in, nitrogen monoxide is removed by a nitrogen monoxide removing agent that is contained in a filter section (not shown).

As the user blows exhalation into the exhalation opening 7, the exhalation that has been blown in passes through the tube 3 and flows into the measurement apparatus main body 4 in FIG. 1. The exhalation contains nitrogen monoxide that has occurred in the airway of the user.

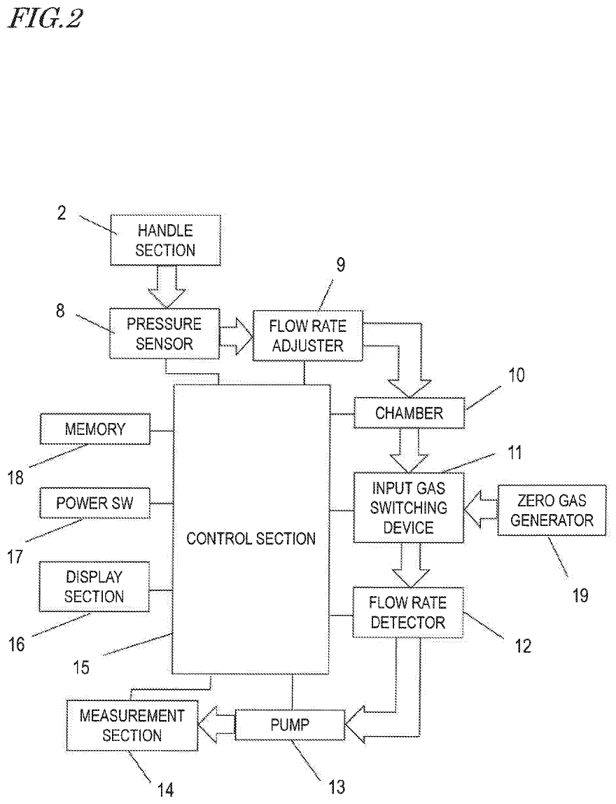

FIG. 2 is a control block diagram of the measurement apparatus main body 4.

As shown in FIG. 2, the following are provided in the measurement apparatus main body 4: a pressure sensor 8 to measure the pressure of the exhalation that is blown in; a flow rate adjuster 9 to adjust the exhalation to a predetermined flow rate (i.e., an amount of exhalation to flow per unit time; e.g., 50 ml/second) by using a value from the pressure sensor 8; and a chamber 10 that temporarily retains the exhalation that has undergone the flow rate adjustment by the flow rate adjuster 9.

Via an input gas switching device 11 and a flow rate detector 12, the exhalation within the chamber 10 is sent from a pump 13 to a measurement section 14. The measurement section 14 takes a measurement of the exhalation.

Note that the pressure sensor 8, the flow rate adjuster 9, the input gas switching device 11, the flow rate detector 12, the pump 13, and the measurement section 14 are electrically connected to a control section 15 as shown in FIG. 2. Also electrically connected to the control section 15 are a display section 16, a power switch 17, and a memory 18. The control section 15 controls the respective sections connected thereto, and also controls operation of the pump 13.

When measuring the nitrogen monoxide contained in the exhalation, after the exhalation is once retained in the chamber 10, the exhalation in the chamber 10 is sucked out by the pump 13 so as to be supplied to the measurement section 14. In other words, when the pump 13 operates, the pressure of the exhalation will be smaller at the pump 13 side than at the chamber 10 side; therefore, as shown in FIG. 2, the exhalation will flow from the chamber 10 toward the measurement section 14.

Now, in order to take a measurement of the exhalation, the exhalation needs to be supplied to the measurement section 14 with a predetermined flow rate.

Therefore, the flow rate detector 12 detects a flow rate of the exhalation that is supplied by the pump 13, and the pump 13 is controlled on the basis of this detected value, whereby the exhalation is supplied to the measurement section 14 with the predetermined flow rate.

This predetermined flow rate is stored in the memory 18 as a predetermined flow rate value that is predefined. Various control programs to be used by the control section 15 are also stored in the memory 18.

Moreover, a zero gas generator 19 is connected to the input gas switching device 11. In an exhalation measurement, the input gas switching device 11 switches from connecting between the chamber 10 and the flow rate detector 12 to connecting between the zero gas generator 19 and the flow rate detector 12.

Hereinafter, a fundamental exhalation measurement operation will be described.

Description herein will begin from a state where the exhalation that has been blown in by the user is already retained in the chamber 10.

In an exhalation measurement, first, the exhalation in the chamber 10 is measured to obtain an actual measurement value.

Specifically, while the control section 15 controls the pump 13 so that the exhalation will have the predetermined flow rate (e.g., 2 ml/second), the exhalation in the chamber 10 is supplied to the measurement section 14, via the input gas switching device 11 and the flow rate detector 12.

At this time, the control section 15 controls the pump 13 on the basis of a detected value of the flow rate of the exhalation as detected by the flow rate detector 12, whereby the exhalation is supplied to the measurement section 14 with the predetermined flow rate.

The measurement section 14 measures the concentration of nitrogen monoxide that is contained in the exhalation. Through this measurement, an actual measurement value of the exhalation is obtained.

Next, the atmospheric air in the measurement environment is measured to obtain a reference value.

Specifically, while the control section 15 controls the pump 13 and the input gas switching device 11, the atmospheric air in the measurement environment is taken in via the zero gas generator 19 and the input gas switching device 11, so as to be supplied to the measurement section 14.

At this time, the atmospheric air in the measurement environment passes through the zero gas generator 19, whereby nitrogen monoxide is removed therefrom. The measurement section 14 measures the atmospheric air in the measurement environment from which nitrogen monoxide has been removed. Through this measurement, a reference value is obtained.

Thereafter, the control section 15 compares actual measurement value of the exhalation against the reference value, thereby calculating an amount of nitrogen monoxide that is contained in the exhalation (i.e., a nitrogen monoxide concentration within the exhalation), and causes this value to be displayed on the display section 16.

In the present embodiment, the detection accuracy for the flow rate of the exhalation as detected by the flow rate detector 12 in FIG. 2 is enhanced, whereby the pump 13 is appropriately controlled on the basis of the detected flow rate of the exhalation, so that the exhalation is supplied to the measurement section 14 with the predetermined flow rate. As a result of this, the measurement accuracy of the concentration of nitrogen monoxide that is contained in the exhalation can be enhanced.

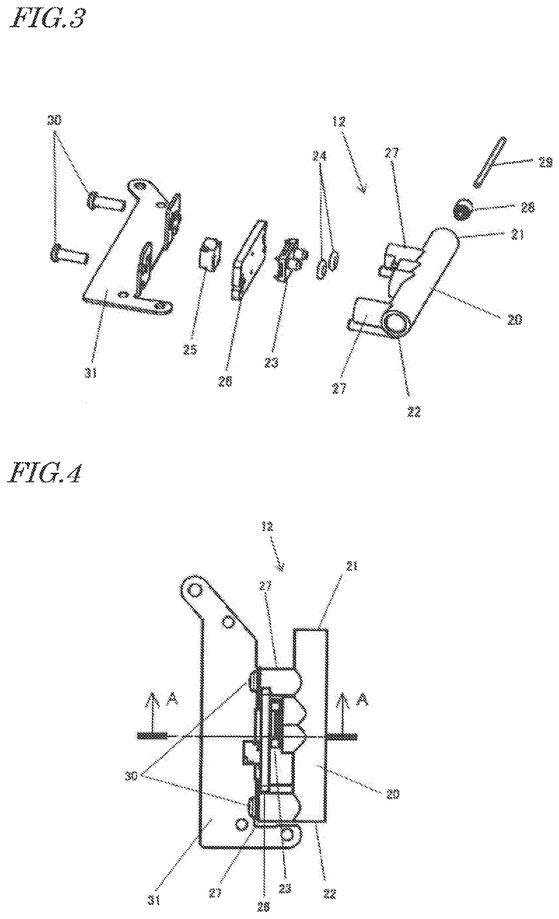

Now, the construction of the flow rate detector 12 in FIG. 2 will be described in detail with reference to FIG. 3 to FIG. 8.

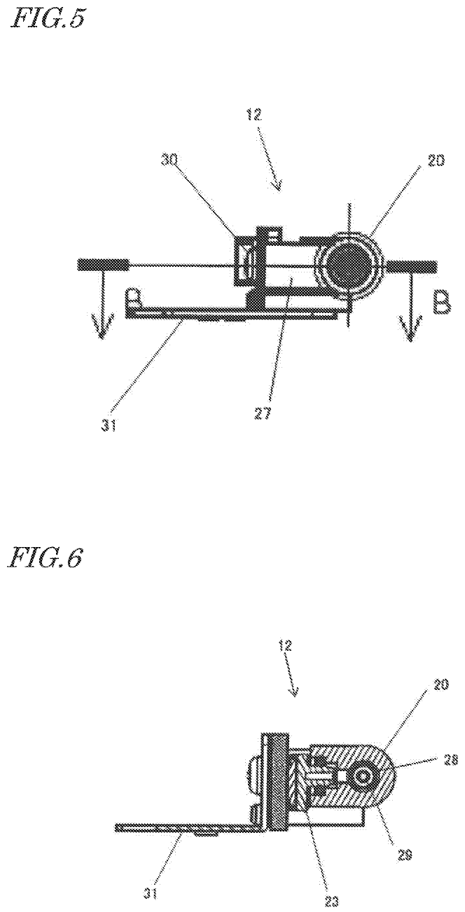

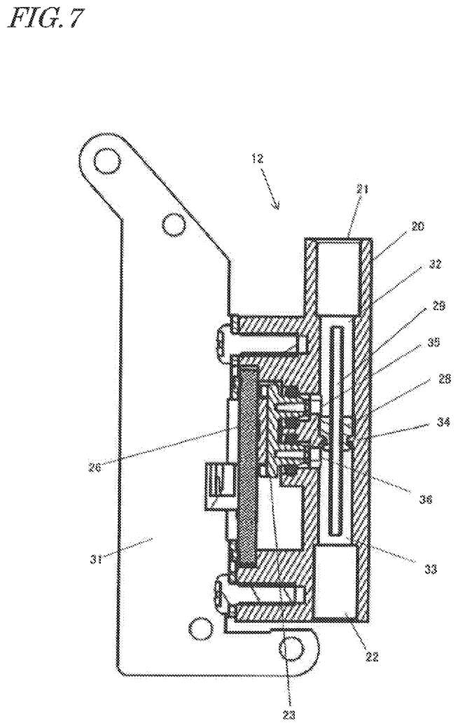

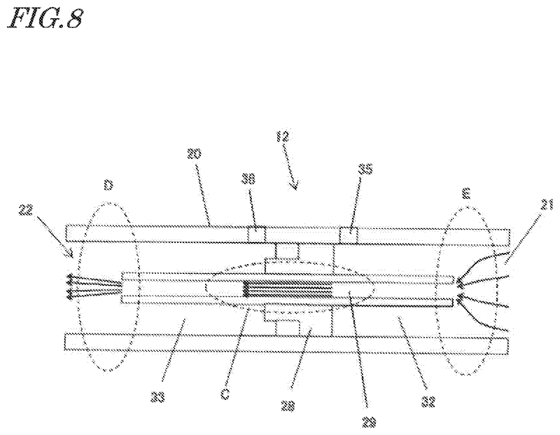

FIG. 3 is an exploded perspective view of the flow rate detector 12. FIG. 4 is an upper plan view of the flow rate detector 12. FIG. 5 is a side view of the flow rate detector 12. FIG. 6 is a cross-sectional view, taken at line A-A and viewed in the direction of arrows in FIG. 4. FIG. 7 is a cross-sectional view, taken at line B-B and viewed in the direction of arrows in FIG. 5. FIG. 8 is a cross-sectional view of a main portion of the flow rate detector 12.

As shown in FIG. 3, the flow rate detector 12 has a pipe body 20 made of resin, having an elongated cylindrical shape.

At a first end, the pipe body 20 has a flow inlet 21 through which exhalation from the chamber 10 (see FIG. 2) flows in. Moreover, at a second end opposite to the first end, the pipe body 20 has a flow outlet 22 through which the exhalation having passed through the pipe body 20 flows out.

Via two O-rings 24, a differential pressure sensor 23 is connected to the pipe body 20. The differential pressure sensor 23 measures pressure at two places, i.e., at the flow-in side and the flow-out side of exhalation, to determine a pressure difference therebetween.

In other words, with respect to the exhalation flowing in the pipe body 20, the exhalation measurement device 1 according to the present embodiment detects a flow rate of the exhalation, from a difference between the pressure measurements taken at the two places by the differential pressure sensor 23.

As the differential pressure sensor 23, a generic differential pressure sensor is employed. The differential pressure sensor 23 is provided on the substrate 26 together with other electronic parts 25.

Moreover, two bosses 27 are provided on the pipe body 20, near the flow inlet 21 and the flow outlet 22.

Furthermore, as will be described in detail later, a single air duct 29 is inserted in the pipe body 20, the air duct 29 being pressed into the center of a partition gasket 28 (which is an example of a partitioning member) having a circular columnar shape.

Then, when the bosses 27 of the flow rate detector 12 are screwed onto a metal plate 31 with two screws 30, the flow rate detector 12 becomes assembled, as shown in FIG. 4 and FIG. 5. The flow rate detector 12 is disposed inside the exhalation measurement device 1.

The inside of the pipe body 20 will be described with reference to FIG. 7.

As shown in FIG. 7, the partition gasket 28 is provided in the pipe body 20, such that the partition gasket 28 partitions the internal space of the pipe body 20 into a flow-in space (first space) 32 on the flow inlet 21 side and a flow-out space (second space) 33 on the flow outlet 22 side.

The partition gasket 28 is a member made of rubber (as an example of an elastic body) that is pressed inside of the pipe body 20, which is substantially cylindrical in shape. A recess which is made at an end of the partition gasket 28 that is closer to the flow outlet 22 is fitted onto an annular rib 34 which protrudes from the inner peripheral surface (inner surface) of the pipe body 20 and inwardly along the radial direction.

As a result of this, the partition gasket 28 can be restrained from moving inside the pipe body 20.

Moreover, the single air duct 29 of metal, having an elongated cylindrical shape, is provided in the partition gasket 28, so as to extend through the central portion of the partition gasket 28.

The air duct 29 is a straight cylindrical member, which is pressed into the partition gasket 28 having a circular columnar shape along its axial direction.

Therefore, the internal space of the pipe body 20 is partitioned into two by the partition gasket 28, such that the partitioned flow-in space 32 and flow-out space 33 communicate with each other via the air duct 29. Therefore, the exhalation which has flown from the flow inlet 21 of the pipe body 20 into the flow-in space 32 passes inside the elongated air duct 29 to reach the flow-out space 33, thus flowing out of the flow outlet 22.

A connection aperture 35 is provided in a portion of the pipe body 20 at a side of the partition gasket 28 that is closer to the flow-in space 32. Through the connection aperture 35, the differential pressure sensor 23 is connected to the flow-in space 32.

On the other hand, a connection aperture 36 is provided in a portion of the pipe body 20 at a side of the partition gasket 28 that is closer to the flow-out space 33. Through the connection aperture 36, the differential pressure sensor 23 is connected to the flow-out space 33.

The differential pressure sensor 23 measures a pressure of the exhalation at each of the flow-in space 32 and the flow-out space 33, and calculates a pressure difference therebetween.

FIG. 8 is a cross-sectional view of the pipe body 20, showing a flow of exhalation inside the pipe body 20.

In the exhalation measurement device 1 according to the present embodiment, as shown in FIG. 8 and as described above, the partition gasket 28 (as an example of a partitioning member) that partitions the internal space of the pipe body 20 into the flow-in space 32 and the flow-out space 33 is provided inside the pipe body 20 composing the flow rate detector 12. Moreover, the single air duct 29 of an elongated shape that extends through the partition gasket 28 is provided.

Moreover, the air duct 29 has a first end 29a protruding from the partition gasket 28 into the flow-in space 32. Similarly, the air duct 29 has a second end 29b opposite to the first end 29a protruding from the partition gasket 28 into the flow-out space 33.

In other words, the exhalation that has flown into the flow-in space 32 is led into the single thin and long air duct 29, which allows the flow-in space 32 and the flow-out space 33 to communicate with each other, and passes through the air duct 29 to flow out into the flow-out space 33.

Therefore, inside the pipe body 20, the exhalation flowing from the flow-in space 32 to the flow-out space 33 moves via the thin and long air duct 29, and consequently the pressure difference between the flow-in space 32 and the flow-out space 33 increases.

Specifically, the pressure at the flow-in space 32, into which the exhalation flows, becomes higher than the pressure at the flow-out space 33, from which the exhalation flows out via the elongated air duct 29. As a result, the pressure difference between the flow-in space 32 and the flow-out space 33 becomes greater than conventional.

Therefore, since the increased pressure difference is measured with a sufficient accuracy by the differential pressure sensor 23, the detection accuracy for the flow rate of the exhalation as detected by using this pressure difference can be enhanced.

As a result, the pump 13 is appropriately controlled by the control section 15 with the enhanced detection accuracy for the flow rate of the exhalation, and the exhalation is supplied to the measurement section 14 with a predetermined flow rate that is managed with a high accuracy. Thus, the measurement accuracy for exhalation can be enhanced.

In the flow-out space 33, as indicated in Area D of FIG. 8, the exhalation that has flown out of the outlet (i.e., the end of the air duct 29 on the flow-out space 33 side) of the air duct 29 gradually expands its flow path, away from the second end 29b at the flow-out side of the air duct 29, this being due to a difference in inner diameter between the pipe body 20 and the air duct 29.

Thus, when a change in the flow path of the exhalation occurs, turbulence (i.e., a flow in which the exhalation irregularly fluctuates with respect to velocity, pressure, etc.) is likely to emerge there. If turbulence emerges near the outlet of the air duct 29, it is foreseeable that the measurement accuracy for pressure difference in the exhalation may lower under the influences of pressure fluctuations due to this turbulence.

Therefore, in the present embodiment, the exhalation is allowed to pass through the elongated air duct 29 as described above. In this respect, the exhalation which has entered the air duct 29 has its direction of flow organized by the elongated air duct 29, and its straightness along the longitudinal direction of the air duct 29 is improved. Then, the exhalation having exited the outlet of the air duct 29 (second end 29b) travels straight over a predetermined distance, whereby changes in the flow path of the exhalation near the outlet of the air duct 29 are suppressed.

This can restrain turbulence from emerging near the outlet of the air duct 29 (Area D of the flow-out space 33).

Then, with the turbulence near the outlet (second end 29b) of the pipe body 20 being suppressed, a pressure difference in the exhalation is measured at a position which is distant from a position where turbulence is likely to emerge (i.e., a position which is distant from the second end 29b at the outlet side of the air duct 29).

Specifically, in the flow-out space 33, the air duct 29 is disposed so as to protrude from the partition gasket 28 into the flow-out space 33, and so as to be distanced from the inner peripheral surface of the pipe body 20. In other words, in the flow-out space 33, a cylindrical gap 37 is created between the outer peripheral surface of the air duct 29 and the inner peripheral surface of the pipe body 20.

The gap 37 is formed at a position which is distant from the second end 29b at the outlet side of the air duct 29, where turbulence is likely to emerge.

Furthermore, the differential pressure sensor 23 to measure a pressure within the flow-out space 33 communicates with the flow-out space 33 via the connection aperture 36 being provided at a position corresponding to the gap 37.

Moreover, the connection aperture 36 is provided at a position which is closer to the partition gasket 28 than to the second end 29b at the outlet side of the air duct 29 in the pipe body 20. Stated otherwise, the connection aperture 36 is provided at a position of the pipe body 20 toward the center of the air duct 29.

As a result, in the flow-out space 33, pressure of the exhalation is measured at a position which is distant from the second end 29b at the outlet side of the air duct 29 (where turbulence is likely to emerge), while turbulence near the outlet of the air duct 29 is suppressed. Therefore, in the flow-out space 33, while hardly being affected by turbulence, pressure of the exhalation can be appropriately measured. Thus, the detection accuracy for the flow rate of the exhalation as detected by using the pressure difference between the flow-out space 33 and the flow-in space 32 can be enhanced.

As a result, the pump 13 is appropriately controlled by the control section 15 with an enhanced detection accuracy for the flow rate of the exhalation, and the exhalation is supplied to the measurement section 14 with a predetermined flow rate that is managed with a high accuracy, whereby the measurement accuracy for exhalation can be enhanced.

Note that, near the inlet of the air duct 29 (i.e., the first end 29a of the air duct 29 at the flow-in space 32 side) in the flow-in space 32, as indicated in Area E of FIG. 8, the exhalation flows into the thin air duct 29.

In the flow-in space 32, too, the air duct 29 is disposed so as to protrude from the partition gasket 28 into the flow-in space 32, and so as to be distanced from the inner peripheral surface of the pipe body 20. In other words, in the flow-in space 32, a cylindrical gap 38 is created between the outer peripheral surface of the air duct 29 and the inner peripheral surface of the pipe body 20.

The gap 38 is formed at a position which is distant from the first end 29a at the inlet side of the air duct 29.

Furthermore, the differential pressure sensor 23 to measure a pressure within the flow-out space 33 communicates with the flow-in space 32, via the connection aperture 35 being provided at a position corresponding to the gap 38.

Moreover, the connection aperture 35 is provided at a position which is closer to the partition gasket 28 than to the first end 29a at the inlet side of the air duct 29 in the pipe body 20. Stated otherwise, the connection aperture 36 is provided at a position of the pipe body 20 toward the center of the air duct 29.

Therefore, even if turbulence emerges near the inlet of the air duct 29 (first end 29a), pressure of the exhalation is measured at a position which is distant from the turbulence. As a result, in the flow-in space 32, pressure of the exhalation can be appropriately measured, while hardly being affected by turbulence. Thus, the detection accuracy for the flow rate of the exhalation by using a pressure difference between the flow-in space 32 and the flow-out space 33 can be enhanced.

As a result, the pump 13 is appropriately controlled by the control section 15 with an enhanced detection accuracy for the flow rate of the exhalation, and the exhalation is supplied to the measurement section 14 with a predetermined flow rate that is managed with a high accuracy, whereby the measurement accuracy for exhalation can be improved relative to the conventional level.

As described above, in the flow-in space 32 and the flow-out space 33, the air duct 29 is so as to be distanced from the inner peripheral surface of the pipe body 20, and substantially cylindrical gaps 37 and 38 are formed between the outer peripheral surface of the air duct 29 and the inner peripheral surface of the pipe body 20.

At the gaps 37 and 38, there is less flow of exhalation (or hardly any flow of exhalation) than there is the flow of exhalation in the air duct 29, the flows of exhalation near the inlet and outlet of the air duct 29 (Area D and Area E of FIG. 8), and the flows of exhalation at the flow inlet 21 and the flow outlet 22 of the pipe body 20.

Furthermore, the differential pressure sensor 23 is connected to the gap 38 of the flow-in space 32 via the connection aperture 35, and connected to the gap 37 of the flow-out space 33 via the connection aperture 36.

As a result, while hardly being affected by unevennesses in the flow of exhalation (e.g., turbulence), pressure difference in the exhalation between the flow inlet 21 and the flow outlet 22 is measured, whereby the detection accuracy for the flow rate of the exhalation can be further enhanced.

Moreover, according to the present embodiment, as shown in FIG. 8, in the flow-out space 33, the connection aperture 36 having the differential pressure sensor 23 connected thereto is provided at a position which is closer to the partition gasket 28 than to the second end 29b at the outlet side of the air duct 29.

In other words, as described above, exhalation is passed through the long air duct in order to suppress turbulence which may emerge near the outlet of the air duct 29; in order to further reduce the influences of turbulence, the connection aperture 36 is disposed at a position which is close to the partition gasket 28, away from the second end 29b at the outlet side of the air duct 29.

Therefore, pressure of the exhalation at the flow-out space 33 side is measured at a position which is less susceptible to influences of turbulence, whereby a pressure difference between the flow-in space 32 and the flow-out space can be measured highly accurately. As a result, the detection accuracy for the flow rate of the exhalation as detected based on a pressure difference between the flow-in space 32 and the flow-out space 33 can be enhanced.

Furthermore, in the present embodiment, as shown in FIG. 8, the air duct 29 is disposed so that its protruding length (i.e., distance from the partition gasket 28 to the first end 29a or the second end 29b of the air duct 29) from the partition gasket 28 is greater than the length (thickness) of the partition gasket 28, along its longitudinal direction.

In particular, in the flow-out space 33, the air duct 29 is formed so that its protruding length from the partition gasket 28 is greater than the length (thickness)of the partition gasket 28, along its longitudinal direction. In other words, the air duct 29 in the flow-out space 33 protrudes far out of the partition gasket 28.

Therefore, the connection aperture 36 to which the differential pressure sensor 23 is connected can be provided at a position which is very distant from a position near the outlet of the air duct 29 where turbulence is likely to emerge (i.e., at a position which is less susceptible to influences of turbulence).

Then, the differential pressure sensor 23 is able to measure the pressure of the exhalation at the flow-out space 33 side, at a position which is less susceptible to influences of turbulence which is likely to emerge near the outlet of the air duct 29.

Thus, since a pressure difference in the exhalation is measured by using a pressure of the exhalation that is measured at a position which is less susceptible to influences of turbulence, the detection accuracy for the flow rate of the exhalation can be further enhanced.

Note that the air duct 29 may be constructed to protrude only into the flow-out space 33. Under this construction, too, providing the connection aperture 36 at a position which is distant from a position near the outlet of the air duct 29 where turbulence is likely to emerge allows the pressure of the exhalation to be measured without being affected by turbulence.

Furthermore, in the present embodiment, the pipe body 20 and the air duct 29 are formed so as to be substantially cylindrical, and disposed so that the center axis of the pipe body 20 and the center axis of the air duct 29 coincide (i.e., overlap). In other words, the pipe body 20 and the air duct 29 constitute a double cylinder in a so-called concentric arrangement.

Therefore, the flow of exhalation in the pipe body 20 can be created in an axisymmetric manner, as centered around the center axis of the pipe body 20 and the air duct 29.

In particular, in the flow-out space 33, the flow of exhalation to come out of the air duct 29 is created in an axisymmetric manner, so that the flow of exhalation is organized in the flow-out space 33, whereby turbulence near the second end 29b of the air duct 29 can be suppressed.

Therefore, a pressure difference in the exhalation between the flow-in space 32 and the flow-out space 33 is measured highly accurately while reducing the influences of turbulence, whereby the detection accuracy for the flow rate of the exhalation can be enhanced.

Furthermore, in the present embodiment, as shown in FIG. 8, the inner diameter of the air duct 29 is smaller than a half of the inner diameter of the pipe body 20.

In other words, a cross-sectional area along a direction which is perpendicular to the axis of the air duct 29 is formed so as to be sufficiently smaller than the cross-sectional area of the pipe body.

Consequently, the exhalation flowing from the flow-in space 32 to the flow-out space 33 is passed through the thin air duct 29, thus resulting in a large pressure difference between the exhalation in the flow-in space 32 and the exhalation in the flow-out space 33.

Therefore, since the increased pressure difference is measured with a sufficient accuracy, the detection accuracy for the flow rate of the exhalation can be enhanced.

Furthermore, in the present embodiment, the inner peripheral surface of the air duct 29 has a smaller surface roughness than does the inner peripheral surface of the pipe body 20.

Specifically, the air duct 29 is made of a metal (e.g., a stainless steel), whereas the pipe body 20 is made of a resin (e.g., an ABS resin).

Accordingly, the inner peripheral surface of the metal air duct 29 has a smaller surface roughness than does the inner peripheral surface of the resin pipe body 20. Therefore, the inner peripheral surface of the air duct 29 has reduced surface irregularities than does the inner peripheral surface of the pipe body 20, thus making it easier for the exhalation to pass through the air duct 29 having reduced surface irregularities.

Thus, the flow of exhalation can be restrained from becoming turbulent within the air duct 29, whereby turbulence near the second end 29b at the outlet side of the air duct 29 (Area D of the flow-out space 33) can be suppressed.

Therefore, a pressure difference in the exhalation between the flow-in space 32 and the flow-out space 33 is measured while reducing the influences of turbulence, whereby the detection accuracy for the flow rate of the exhalation as detected by using this pressure difference can be further enhanced.

Furthermore, in the present embodiment, in the flow-out space 33, the connection aperture 36 having the differential pressure sensor 23 connected thereto is provided at a position which is closer to the center of the air duct 29 (i.e., toward the partition gasket 28) than to the second end 29b of the air duct 29.

In other words, in the present embodiment, exhalation is passed through the long air duct 29 in the aforementioned manner, thus enhancing straightness of the exhalation, and suppressing turbulence which may emerge near the outlet of the air duct 29; in order to further reduce the influences of turbulence, the connection aperture 36 is provided at a position which is close to the center of the air duct 29, away from the second end 29b at the outlet side of the air duct 29.

Therefore, pressure of the exhalation at the flow-out space 33 side is measured at a position which is distant from a position where turbulence is likely to emerge, whereby the detection accuracy for the flow rate of the exhalation can be further enhanced.

Furthermore, in the present embodiment, the air duct 29 is made of a single cylinder.

That is, when the air duct 29 is formed by bundling together a multitude of elongated tubes, the inner peripheral surface of each tube will have a small machining error. Before and after the air duct 29, machining errors of a plurality of tubes will add up and increase, thus allowing the pressure of the exhalation to fluctuate. This might lower the detection accuracy for the flow rate of the exhalation.

In the exhalation measurement device 1 according to the present embodiment, the air duct 29 is made of a single cylinder, and therefore machining errors will not add up.

Thus, since the pressure difference in the exhalation is appropriately measured, the detection accuracy for the flow rate of the exhalation using this pressure difference can be further enhanced.

Note that, in the present embodiment, both of the connection aperture 35 at the inlet side of the air duct 29 and the connection aperture 36 at the outlet side of the air duct 29 are provided at positions respectively corresponding to the gaps 37 and 38 created between the outer peripheral surface of the air duct 29 and the inner peripheral surface of the pipe body 20, as described above.

However, when the turbulence emerging near the inlet of the air duct 29 is smaller than the turbulence near the outlet of the air duct 29, it suffices if only the connection aperture 36 at the outlet side is provided at the position corresponding to the gap 37 between the air duct 29 and the pipe body 20.

In other words, in the case where the flow of exhalation that reaches the inlet of the air duct 29 of the flow-in space 32 is a laminar flow (i.e., a flow in which the exhalation does not irregularly fluctuate with respect to velocity, pressure, etc.), for example, the turbulence emerging near the inlet of the air duct 29 (first end 29a) will be negligibly small relative to the turbulence emerging near the outlet (second end 29b).

In this case, only the connection aperture 36 at the outlet (second end 29b) side may be provided at the position corresponding to the gap 37 created between the outer peripheral surface of the air duct 29 and the inner peripheral surface of the pipe body 20.

Thus, the exhalation measurement device 1 according to the present embodiment includes: a measurement apparatus main body 4 into which exhalation is to be blown; a measurement section 14 to take a measurement of the exhalation; a chamber 10 to temporarily retain the exhalation that has been blown into the measurement apparatus main body 4; a pump 13 to allow the exhalation in the chamber 10 to be supplied to the measurement section 14; a control section 15 to control operation of the pump 13; and a flow rate detector 12 to detect a flow rate of the exhalation that is supplied by the pump 13 to the measurement section 14.

The flow rate detector 12 includes: a pipe body 20 having a flow inlet 21 at a first end, into which the exhalation flows, and a flow outlet 22 at a second end, out of which the exhalation flows; and a differential pressure sensor 23 connected to the pipe body 20.

The pipe body 20 includes: a partition gasket 28 (as an example of a partitioning member) which partitions the inside of the pipe body 20 into a flow-in space 32 and a flow-out space 33 for the exhalation; a connection aperture 35 being provided at the flow-in space 32 and connecting the differential pressure sensor 23 to the flow-in space 32; a connection aperture 36 being provided at the flow-out space 33 and connecting the differential pressure sensor 23 to the flow-out space 33; and an air duct 29 of an elongated shape extending through the partition gasket 28 and allowing the flow-in space 32 and the flow-out space 33 to communicate with each other.

Thus, in the exhalation measurement device 1 according to the present embodiment, the pipe body 20 of the flow rate detector 12 includes: the partition gasket 28, which partitions the inside into the flow-in space 32 and the flow-out space 33 for the exhalation; and the air duct 29 of an elongated shape, extending through the partition gasket 28 and allowing the flow-in space 32 and the flow-out space 33 to communicate with each other.

Therefore, inside the pipe body 20, the exhalation passes through the thin and long air duct 29 in moving over to the flow-out space 33 side, thus inducing a large pressure difference in the exhalation between the flow-in space 32 and the flow-out space 33.

Accordingly, a pressure difference which has been made greater than conventional is measured with a sufficient accuracy, whereby the detection accuracy for the flow rate of the exhalation as detected by using this pressure difference can be enhanced.

As a result, the pump 13 is appropriately controlled so as to attain a flow rate of the exhalation which is highly accurately managed on the basis of the accurately detected pressure difference, whereby the measurement accuracy of the concentration of nitrogen monoxide that is contained in the exhalation can be enhanced.

### INDUSTRIAL APPLICABILITY

The present invention is expected to find use in an exhalation measurement device to be used when e.g. detecting asthma, or checking pulmonary function.

### REFERENCE SIGNS LIST

1 exhalation measurement device
2 handle section
3 tube
4 measurement apparatus main body
5 handle section main body
6 mouthpiece
7 exhalation opening
8 pressure sensor
9 flow rate adjuster
10 chamber
11 input gas switching device
12 flow rate detector
13 pump
14 measurement section
15 control section
16 display section
17 power switch
18 memory
19 zero gas generator
20 pipe body
21 flow inlet
22 flow outlet
23 differential pressure sensor
24 O-ring
25 electronic parts
26 substrate
27 boss
28 partition gasket (partitioning member)
29 air duct
29a first end
29b second end
30 screw
31 metal plate
32 flow-in space (first space)
33 flow-out space (second space)
34 rib
35 connection aperture
36 connection aperture
37 gap
38 gap

1. An exhalation measurement device comprising:
a measurement apparatus main body into which exhalation is to be blown;
a measurement section to take a measurement of the exhalation;
a chamber to temporarily retain the exhalation that has been blown into the measurement apparatus main body;
a pump to allow the exhalation in the chamber to be supplied to the measurement section;
a control section to control operation of the pump; and
a flow rate detector to detect a flow rate of the exhalation that is supplied by the pump to the measurement section, wherein,
the flow rate detector includes
a pipe body having a flow inlet at a first end, into which the exhalation flows, and a flow outlet at a second end opposite to the first end, out of which the exhalation flows, and
a differential pressure sensor connected to the pipe body; and
the pipe body includes
a partitioning member which partitions an inside of the pipe body into a first space at a flow-in side of the exhalation and a second space at a flow-out side of the exhalation,
a first connection aperture being provided at the first space side and connecting the differential pressure sensor to the first space,
a second connection aperture being provided at the second space side and connecting the differential pressure sensor to the second space, and
an air duct of an elongated shape extending through the partitioning member and allowing the first space and the second space to communicate with each other.

2. The exhalation measurement device of claim 1, wherein,
in the second space,
the air duct is disposed so as to be distanced from an inner surface of the pipe body and protruding from the partitioning member, and
the second connection aperture is disposed at a position corresponding to a gap created between an outer surface of the air duct and the inner surface of the pipe body.

3. The exhalation measurement device of claim 2, wherein,
in the first space,
the air duct is disposed so as to be distanced from the inner surface of the pipe body and protruding from the partitioning member, and
the first connection aperture is disposed at a position corresponding to a gap created between the outer surface of the air duct and the inner surface of the pipe body.

4. The exhalation measurement device of claim 2, wherein,
in the second space,
the second connection aperture is provided at a position which is closer to the partitioning member than to an end of the air duct along a longitudinal direction thereof.

5. The exhalation measurement device of claim 1, wherein,
in the second space,
the air duct is formed so that, along a longitudinal direction thereof, a protruding length from the partitioning member is greater than a length of the partitioning member.

6. The exhalation measurement device of claim 1, wherein,
the pipe body is formed so as to be substantially cylindrical;
the air duct is formed so as to be substantially cylindrical; and
a center axis of the pipe body and a center axis of the air duct are disposed so as to overlap each other.

7. The exhalation measurement device of claim 6, wherein an inner diameter of the air duct is smaller than a half of an inner diameter of the pipe body.

8. The exhalation measurement device of claim 1, wherein an inner surface of the air duct has a smaller surface roughness than does an inner surface of the pipe body.

9. The exhalation measurement device of claim 1, wherein,
in the second space,
the second connection aperture is provided at a position which is closer to the center of the air duct than to an end of the air duct along a longitudinal direction thereof.

10. The exhalation measurement device of claim 1, wherein the air duct is made of a single cylinder.
